(19) United States
(12) Patent Application Publication
(10) Pub. No.: US 2009/0171111 A1
(43) Pub. Date: Jul. 2, 2009

(54) Method for Producing Sodium Chloride-Free Ammonium Nitriles

(76) Inventors: Lars Cuypers, Muenchen (DE); Jean Hypolites Koek, Schiedam (NL)

Correspondence Address: CLARIANT CORPORATION;INTELLECTUAL PROPERTY DEPARTMENT, 4000 MONROE ROAD, CHARLOTTE, NC, 28205, US

(21) Appl. No.: 12/225,521

(22) PCT Filed: Mar. 16, 2007
(86) PCT No.: PCT/EP2007/002329

§ 371 Date: Sep. 23, 2008

(30) Foreign Application Priority Data
Mar. 24, 2006 (DE) 10 2006 013 666.7

(51) Int. Cl. C07C 255/24

(52) U.S. Cl. 558/452

(57) ABSTRACT

This invention relates to a method for producing sodium chloride-free compounds of formula (I),

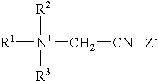

wherein R¹, R², R³ and Z are defined in the description. The method according to the invention is characterized by reacting a tertiary amine of the formula NR¹R²R³ with chloroacetonitrile in an organic solvent and then adding an acid of the formula HZ.

This invention relates to an improved process for preparing ammonionitriles with low hygroscopicity by reacting a tertiary amine with chloroacetonitrile in an organic solvent and then adding a strong acid, for example toluenesulfonic acid.

Inorganic peroxygen compounds, especially hydrogen peroxide and solid peroxygen compounds which dissolve in water with release of hydrogen peroxide, such as sodium perborate and sodium carbonate perhydrate, have been used for some time as oxidizing agents for disinfection and bleaching purposes. The oxidizing action of these substances in dilute solutions depends greatly on the temperature; for example, with hydrogen peroxide or perborate in alkaline bleaching liquors, sufficiently rapid bleaching of soiled textiles is achieved only at temperatures above about 80° C.

It is known that the oxidizing action of peroxidic bleaches, such as perborates, percarbonates, persilicates and perphosphates, can be improved at low temperatures by adding precursors of bleaching peroxy acids, known as bleach activators. Many substances are known to be bleach activators according to the prior art. Usually, these are reactive organic compounds with an O-acyl or N-acyl group which form the corresponding peroxy acids in alkaline solution together with a source for hydrogen peroxide. Representative examples of bleach activators are, for instance, N,N,N′,N′-tetraacetylethylenediamine (TAED), glucose pentaacetate (GPA), xylose tetraacetate (TAX), sodium-4-benzoyloxy-benzenesulfonate (SBOBS), sodium trimethylhexanoyloxy-benzenesulfonate (STHOBS), tetraacetylglycoluril (TAGU), tetraacetylcyanic acid (TACA), di-N-acetyldimethylglyoxime (ADMG), 1-phenyl-3-acetylhydantoin (PAH), sodium nonanoyloxybenzenesulfonate (NOBS) and sodium isononanoyloxybenzenesulfonate (ISONOBS).

Addition of these substances allows the bleaching action of aqueous peroxide solutions to be enhanced to such an extent that, even at temperatures around 60° C., essentially the same effects occur as with the peroxide solution alone at 95° C.

Some cationic compounds which contain a quaternary ammonium group have become more significant, since they are highly effective bleach activators. Such cationic bleach activators are described, for example, in GB-A-1 382 594, U.S. Pat. No. 4,751,015, EP-A-0 284 292, EP-A-0 331 229.

Ammonionitriles of the formula

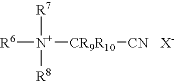

form an exceptional class of cationic bleach activators. Compounds of this type and the use thereof as activators in bleaches are described, for example, in EP-A-303 520, EP-A-458 396 and EP-A-464 880. In the perhydrolysis, these compounds probably form a peroxyimide acid which acts as the bleaching agent.

It is known that many ammonionitriles which possess a halide as the counterion X⁻ have a high hygroscopicity and are therefore unsuitable for use in solid washing and cleaning compositions. EP-A-0464880 describes ammonionitriles of the general formula

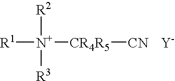

where R₄ and R₅ are each individually H or a substituent group which contains at least one carbon atom; R¹ is straight- or branched-chain and is a C₁-C₂₄-alkyl, -alkenyl or -alkyl ether group or CR₄R₅CN; R² and R³ are each individually a C₁-C₄-alkyl or -hydroxyalkyl group; or R¹ is also a group of the formula:

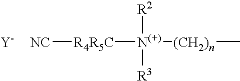

in which n is an integer of 1to about 4; and Y⁻ is the counterion, selected from the group of 1) R—SO₃⁻, 2) R—SO₄⁻, 3) R—CO₂⁻, where R is a straight- or branched-chain, optionally substituted alkyl, alkyl ether or alkylene group which contains from 4 to 20 carbon atoms, or a phenyl or alkylphenyl group which contains from 6 to 20 carbon atoms, and 4) surfactant anions which do not fall within group 1), 2) and 3). Compared to the compounds with halide counterions, these compounds exhibit significantly lower hygroscopicity. EP-A-0464880 describes three preparation routes for the synthesis of the compounds mentioned: two direct syntheses using methylsulfonates or methyl sulfates as the alkylating quaternizing reagents and one anion exchange in alcoholic solvents. The anion exchange reactions in alcoholic solvents described in example I in EP-A-0464880 have a remarkably high consumption of solvents and energy; for instance, in example c, to prepare 3.4 g of product, first 100 ml of methanol are added and distilled off and then 150 ml of isopropanol are added and again distilled off. This procedure is ecologically and economically unviable for industrial scale processes. Moreover, it is not possible through this anion exchange reaction to isolate a sodium chloride-free product.

It is therefore an object of the invention to develop a process which can be performed both on the industrial scale and continuously, which leads, in very good yields and with an acceptable level of complexity, to products which, with regard to composition, quality and color, are suitable for use in washing and cleaning compositions and are free of sodium chloride.

It has now been found that, surprisingly, ammonionitriles of the type described above can be prepared in a very simple manner by the reaction of the corresponding tertiary amines with chloroacetonitrile in organic solvents and subsequent addition of strong acids, for example toluenesulfonic acid.

The present invention therefore provides a process for preparing compounds of the general formula (I)

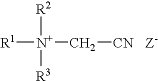

where R¹ is a straight- or branched-chain C₁-C₂₄-alkyl, C₂-C₂₄-alkenyl or C₁-C₂₄-alkyl ether group or —CH₂CN or a group of the formula

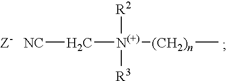

R² and R³ are each individually a C₁-C₈-alkyl or C₁-C₄-hydroxyalkyl group;
n is an integer from 1 to 4; and Z⁻ is a counterion of the formula R—SO₃⁻ or R—SO₄⁻, where R is a straight- or branched-chain, optionally substituted alkyl, alkyl ether or alkylene group which contains from 4 to 20 carbon atoms, or a phenyl or alkylphenyl group which contains a total of from 6 to 20 carbon atoms, or Z⁻ is a counterion of the formula PF₆⁻, ClO₄⁻, NO₃⁻ or a perfluorinated alkanesulfonate or perfluorinated alkanecarboxylate, which comprises reacting a tertiary amine of the formula NR¹R²R³ with chloroacetonitrile in an organic solvent and then adding an acid of the formula HZ.

The invention relates both to compounds of the abovementioned general formula (I) in which R¹ is a straight- or branched-chain C₁-C₄-alkyl, C₂-C₄-alkenyl or C₁-C₄-alkyl ether group or —CH₂CN group, and R² and R³ are each individually a C₁-C₄-alkyl or C₁-C₄-hydroxyalkyl group,

and to compounds of the formula (I), in which R¹ is a group of the formula

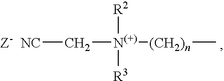

and R² and R³ are each individually a C₁-C₄-alkyl or C₁-C₄-hydroxyalkyl group and n is an integer from 1 to 4,
and to compounds of the formula (I) in which R¹ is a C₅-C₂₄-alkyl, C₅-C₂₄-alkenyl or C₅-C₂₄-alkyl ether group, and R² and R³ are each individually a C₁-C₈-alkyl or C₁-C₄-hydroxyalkyl group.

The tertiary amines which serve as the starting compound are preferably compounds of the formula NR¹R²R³ in which R¹ is C₁- to C₂₄-alkyl and R² and R³ are each independently C₁- to C₆-alkyl, or diamines of the formula

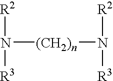

in which R² and R³ are each independently C₁- to C₆-alkyl.

The tertiary amines and the diamines may be pure substances or mixtures of different amines of different carbon chain lengths.

The counterions Z⁻ are preferably perfluorinated alkanesulfonates such as CF₃SO₃⁻ or perfluorinated alkanecarboxylate such as CF₃CO₂⁻, C₁₂-C₁₈-alkane- or paraffinsulfonate, primary C₁₂-C₁₈ alcohol sulfate or C₁-C₄-alkylbenzenesulfonate. Particular preference is given to the tosylate ion in the definition of Z.

The organic solvents used are preferably ketones, alkyl acetates, aromatic hydrocarbons such as toluene, xylene or cumene, alkanes having a boiling point of >30° C., di- or trichloromethane, N-methylpyrrolidone, acetonitrile, 1,3-dimethylimidazolidin-2-one, N,N-dimethylacetamide, dimethyl sulfoxide, dimethylformamide, ethers such as methyl tert-butyl ether or tetra-hydrofuran or mixtures of these solvents. Tertiary monoamine and chloroacetonitrile are reacted with one another in a ratio of from 0.9:1 to 2:1, preferably from 1:1 to 1.5:1. Tertiary diamine and chloroacetonitrile are reacted with one another in a ratio of from 1:1 to 1:4, preferably from 1:1.5 to 1:2.5. The acid HZ is added in a ratio of from 0.5:1 to 2:1, preferably from 0.75:1 to 1.5:1 based on the tertiary monoamine, or in a ratio of from 1:1 to 4:1, preferably from 1.5:1 to 2.5:1, based on the tertiary diamine.

The reaction of the amine with chloroacetonitrile is carried out at temperatures between 25 and 150° C., preferably 30-100° C. The addition of the acid HZ is carried out at temperatures between 25 and 150° C., preferably 30-120° C. The product is isolated at temperatures between −30 and 50° C., preferably −10 and 30° C.

The acid HZ can be added in solid or liquid form or in the form of a suspension or solution based on the organic solvent. The acid HZ should preferably be anhydrous on addition, in order that there is no partial hydrolysis of the nitrile compound. If appropriate, the acid should be dried before the addition, as known to those skilled in the art.

The total reaction time is guided by the reaction conditions and may be between 1 and 24 hours, preferably from 2 to 10 hours. In a particular embodiment, the process according to the invention can be performed continuously. Particularly suitable for this purpose are stirred tank batteries and tubular reactors, as are known to those skilled in the art. After the reaction has ended, the reaction product is isolated by means of conventional separation methods. Suitable apparatus for this purpose includes centrifuges or filter apparatus. For the purification of the end product, it is advisable to extract the crude reaction product by washing once or more than once with the reaction medium or the solvent. The mother liquor can optionally be used for the next reaction without purification, i.e. recycled.

The hydrogen chloride formed is removed from the reaction mixture via the gas phase, if appropriate while purging with dried inert gases, for example dry nitrogen, and if appropriate while lowering the pressure. The offgases should subsequently be washed, as known to those skilled in the art.

The ammonionitrile formed is obtained in high yields in the form of a colorless powder which can be isolated by conventional drying.

The advantage of the process according to the invention lies in the fact that the hydrolysis-stable sulfate or sulfonate salts can be prepared without the product being contaminated with chloride and alkali metal ions.

The ammonionitrile obtained in this way can be used as a bleach activator in washing and cleaning compositions such as pulverulent or tableted heavy-duty washing compositions, stain removal salts or pulverulent machine dishwasher detergents. To increase the storage stability in these formulations, it can be converted to a granular form, as known to those skilled in the art.

### EXAMPLES

### Example 1

### Preparation of Anhydrous Toluenesulfonic Acid

In a flask with a water separator, 190.2 g (1 mol) of toluene-4-sulfonic acid monohydrate were stirred under reflux in 600 ml of toluene for 3 hours. A total of approx. 18 ml of water were separated out. The solution of toluene-4-sulfonic acid in toluene was used for the subsequent anion exchange reaction without further workup.

### Example 2

### Synthesis of (cyanomethyl)diethylmethylammonium Tosylate

87.2 g (1 mol) of diethylmethylamine were initially charged at 50° C. in 1000 ml of toluene, and 75.5 g (1 mol) of chloroacetonitrile were added. The reaction mixture was stirred at 60° C. for 3 hours. Then 172.2 g of anhydrous toluenesulfonic acid dissolved in 600 ml of toluene were added at 25° C. and the reaction mixture was stirred under reflux for 4 hours, in the course of which evolution of gas was observed. The reaction mixture was cooled slowly to 5° C., and the precipitated solid was filtered, washed twice with 100 ml each time of toluene and dried at 60° C. under reduced pressure.

245.9 g (0.82 mol) of (cyanomethyl)diethylmethylammonium tosylate were obtained as a colorless solid, corresponding to a yield of 82%.

¹H NMR (D₂O): δ=7.70 (2H, d); δ=7.36 (2H, d); δ=4.62 (2H, s); δ=3.54 (4H, q); δ=3.17 (3H, s); δ=2.39 (3H, s); δ=1.37 (6H, t).

### Example 3

### Synthesis of (cyanomethyl)diisopropylmethylammonium Tosylate

115.2 g (1 mol) of diisopropylmethylamine were initially charged at 50° C. in 1000 ml of toluene, and 75.5 g (1 mol) of chloroacetonitrile were added. The reaction mixture was stirred at 60° C. for 3 hours. Then 172.2 g of anhydrous toluenesulfonic acid dissolved in 600 ml of toluene were added at 25° C. and the reaction mixture was stirred under reflux for 4 hours, in the course of which evolution of gas was observed. The reaction mixture was cooled slowly to 5° C., and the precipitated solid was filtered, washed twice with 100 ml each time of toluene and dried at 60° C. under reduced pressure.

201.5 g (0.63 mol) of (cyanomethyl)diisopropylmethylammonium tosylate were obtained as a colorless solid, corresponding to a yield of 63%.

¹H NMR (D₂O): δ=7.65 (2H, d); δ=7.32 (2H, d); δ=4.75 (2H, s); δ=4.13 (2H, m); δ=2.97 (3H, s); δ=2.34 (3H, s); δ=1.47 (6H, d); δ=1.42 (6H, d).

### Example 4

### Synthesis of (cyanomethyl)dimethyloctylammonium Tosylate

157.3 g (1 mol) of dimethyloctylamine were initially charged at 50° C. in 1000 ml of toluene, and 75.5 g (1 mol) of chloroacetonitrile were added. The reaction mixture was stirred at 60° C. for 3 hours. Then 172.2 g of anhydrous toluenesulfonic acid dissolved in 600 ml of toluene were added at 25° C. and the reaction mixture was stirred under reflux for 4 hours, in the course of which evolution of gas was observed. The reaction mixture was cooled slowly to 5° C., and the precipitated solid was filtered, washed twice with 100 ml each time of toluene and dried at 60° C. under reduced pressure.

289.9 (0.79 mol) of (cyanomethyl)dimethyloctylammonium tosylate were obtained as a colorless solid, corresponding to a yield of 79%.

¹H NMR (D₂O): δ=7.70 (2H, d); δ=7.37 (2H, d); δ=4.75 (2H, s); δ=3.56 (2H, m); δ=3.33 (6H, s); δ=2.40 (3H, s); δ=1.85 (2H, m); δ=1.45-1.26 (10H, m); δ=0.89 (3H, t).

### Example 5

### Synthesis of (cyanomethyl)dimethyldodecylammonium Tosylate

213.4 g (1 mol) of dimethyldodecylamine were initially charged at 50° C. in 1000 ml of toluene, and 75.5 g (1 mol) of chloroacetonitrile were added. The reaction mixture was stirred at 60° C. for 3 hours. Then 172.2 g of anhydrous toluenesulfonic acid dissolved in 600 ml of toluene were added at 25° C. and the reaction mixture was stirred under reflux for 4 hours, in the course of which evolution of gas was observed. The reaction mixture was cooled slowly to 5° C., and the precipitated solid was filtered, washed twice with 100 ml each time of toluene and dried at 60° C. under reduced pressure.

357.7 g (0.84 mol) of (cyanomethyl)dimethyldodecylammonium tosylate were obtained as a colorless solid, corresponding to a yield of 84%.

¹H NMR (D₂O): δ=7.70 (2H, d); δ=7.37 (2H, d); δ=4.75 (2H, s); δ=3.54 (2H, t); δ=3.33 (6H, s); δ=2.40 (3H, s); δ=1.84 (2H, m); δ=1.40-1.25 (18H, m); δ=0.88 (3H, t).

### Example 6

### Synthesis of (cyanomethyl)dihexylmethylammonium Tosylate

199.4 g (1 mol) of dihexylmethylamine were initially charged at 50° C. in 1000 ml of butyl acetate, and 75.5 g (1 mol) of chloroacetonitrile were added. The reaction mixture was stirred at 60° C. for 3 hours. Then 172.2 g of anhydrous toluenesulfonic acid dissolved in 500 ml of butyl acetate were added at 25° C. and the reaction mixture was stirred under reflux for 4 hours, in the course of which evolution of gas was observed. The reaction mixture was cooled slowly to 5° C., and the precipitated solid was filtered, washed twice with 100 ml each time of butyl acetate and dried at 60° C. under reduced pressure.

319.2 g (0.78 mol) of (cyanomethyl)dihexylmethylammonium tosylate were obtained as a colorless solid, corresponding to a yield of 78%.

¹H NMR (D₂O): δ=7.70 (2H, d); δ=7.37 (2H, d); δ=4.75 (2H, s); δ=3.52 (4H, t); δ=3.28 (3H, s); δ=2.40 (3H, s); δ=1.81 (4H, m); δ=1.42-1.27 (12H, m); δ=0.90 (6H, t).

1. A process for preparing sodium chloride-free compounds of the general formula (I) 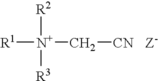
where R¹ is a straight- or branched-chain C₁-C₂₄-alkyl, C₂-C₂₄-alkenyl or C₁-C₂₄-alkyl ether group or —CH₂CN or a group of the formula 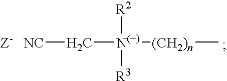
R² and R³ are each individually a C₁-C₈-alkyl or C₁-C₄-hydroxyalkyl group;
n is an integer of 1 to 4; and Z⁻ is a counterion of the formula R—SO₃⁻ or R—SO₄⁻, where R is a straight- or branched-chain, optionally substituted alkyl, alkyl ether or alkylene group which contains from 4 to 20 carbon atoms, or a phenyl or alkylphenyl group which contains a total of from 6 to 20 carbon atoms, or Z⁻ is a counterion of the formula PF₆⁻, ClO₄⁻, NO₃⁻ or a perfluorinated alkanesulfonate or perfluorinated alkanecarboxylate, said process comprising reacting a tertiary amine of the formula NR¹R²R³ with chloroacetonitrile in an organic solvent selected from the group consisting of acetone, butanone, pentanone, hexanone, cyclohexanone, methyl isobutyl ketone, alkyl acetate, toluene, xylene, cumene, hexane, heptane, octane, dichloromethane, trichloromethane, dimethyl sulfoxide, N-methylpyrrolidone, 1,3-dimethylimidazolidin-2-one, dimethylformamide, N,N-dimethylacetamide, acetonitrile and mixtures thereof to provide an intermediate and then adding an acid of the formula HZ.

2. The process as claimed in claim 1, wherein the compound of the formula (I) is prepared, in which R¹ is a straight- or branched-chain C₁-C₄-alkyl, C₂-C₄-alkenyl or C₁-C₄-alkyl ether group or —CH₂CN, and R² and R³ are each individually a C₁-C₄-alkyl or C₁-C₄-hydroxyalkyl group, and Z⁻ is a counterion of the formula PF₆⁻, ClO₄⁻, NO₃⁻ or a perfluorinated alkanesulfonate or perfluorinated alkanecarboxylate.

3. The process as claimed in claim 1, wherein the compound of the formula (I) is prepared, in which R¹ is a group of the formula 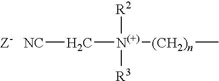
and R² and R³ are each individually a C₁-C₄-alkyl or C₁-C₄-hydroxyalkyl group, and Z⁻ is a counterion of the formula PF₆⁻, ClO₄⁻, NO₃⁻ or a perfluorinated alkanesulfonate or perfluorinated alkanecarboxylate.

4. The process as claimed in claim 1, wherein the compound of the formula (I) is prepared, in which R¹ is a straight- or branched-chain C₅-C₂₄-alkyl, C₅-C₂₄-alkenyl or C₅-C₂₄-alkyl ether group, and R², R³, n and Z⁻ is a counterion of the formula PF₆⁻, ClO₄⁻, NO₃⁻ or a perfluorinated alkanesulfonate or perfluorinated alkanecarboxylate.

5. The process as claimed in claim 1, wherein the intermediate from the reaction of the amine with chloroacetonitrile is not isolated or purified.

6. The process as claimed in claim 1, wherein the acid HZ is added in the form of a solution or suspension based on the organic solvent.

7. The process as claimed in claim 1, wherein the compound of the formula (I) is prepared, in which R¹ is C₁- to C₁₈-alkyl.

8. The process as claimed in claim 1, wherein the compound of the formula (I) is prepared, in which R² and R³ are each individually a C₁-C₆-alkyl group.

9. The process as claimed in claim 1, wherein the compound of the formula (I) is prepared, in which Z⁻ is alkane- or paraffinsulfonate, primary C₁₂-C₁₈ alcohol sulfate or optionally substituted benzenesulfonate.

10. The process as claimed in claim 1, wherein the acid HZ used is an acid which has a water content of less than 1% by weight.

11. The process as claimed in claim 1, wherein the acid HZ used is an acid which has a pKa of <−5.
